(19) United States
(12) Patent Application Publication
(10) Pub. No.: US 2018/0193643 A1
(43) Pub. Date: Jul. 12, 2018

(54) METHOD AND DEVICE FOR SACRAL NERVE STIMULATION

(71) Applicant: The Johns Hopkins University, Baltimore, MD (US)

(72) Inventors: Jiande Chen, Baltimore, MD (US); Jieyun Yin, Baltimore, MD (US); Pankaj J. Pasricha, Baltimore, MD (US)

(21) Appl. No.: 15/579,488

(22) PCT Filed: Jun. 3, 2016
(86) PCT No.: PCT/US2016/035882

§ 371 Date: Mar. 29, 2018

Related U.S. Application Data
(60) Provisional application No. 62/171,488, filed on Jun. 5, 2015.

(51) Int. Cl. A61N 1/36

(52) U.S. Cl. CPC A61N 1/36053; A61N 1/36135; A61N 1/36189; A61N 1/36132; A61N 1/36146

(57) ABSTRACT

The present invention provides a method and device for sacral nerve stimulation (SNS) for treatment of disease, such as inflammatory disease. The SNS therapy is automatically adjusted in response to detection of physiological data from the patient.

### CROSS REFERENCE TO RELATED APPLICATION

This application claims the benefit of priority under 35 U.S.C. § 119(e) of U.S. Provisional Patent Application Ser. No. 62/171,488, filed Jun. 5, 2015, the entire contents of which is incorporated herein by reference in its entirety.

### BACKGROUND OF THE INVENTION

### Field of the Invention

The present disclosure relates generally to medical devices and more specifically to a method and device for providing nerve stimulation to treat disease.

### Background Information

About 5%-7% of general population in Western society is affected by immune-mediated inflammatory diseases (1) that include, but not limited to, ankylosing spondylitis, psoriasis, psoriatic arthritis, rheumatoid arthritis, and inflammatory bowel disease (IBD). The IBD includes Crohn's disease (CD) and ulcerative colitis (UC). The prevalence of inflammatory diseases is even higher in veterans population due to frequent exposures to stress. Although these diseases have different epidemiologies and pathophysiologies, they have one thing in common: an imbalance in inflammatory cytokines. A cholinergic anti-inflammatory pathway has recently been implicated. Animal studies have shown a preventive effect of vagal nerve stimulation (VNS) on inflammation in the gut and a recent clinical study has demonstrated a therapeutic role of VNS for rheumatoid arthritis.

A sacral nerve stimulator is a device used to provide electrical stimulation to the pelvic region of a patient, for example the sacral nerve, in order to treat problems such as incontinence. Stimulators typically include an implanted or external pulse generator and an implanted stimulation lead having one or more electrodes at a distal location thereof. The pulse generator provides the stimulation through the electrodes via a body portion and connector of the lead. Stimulation programming in general refers to the configuring of stimulation electrodes and stimulation parameters to treat the patient using one or more implanted leads and its attached implantable pulse generator (IPG). For example, the programming is typically achieved by selecting individual electrodes and adjusting the stimulation parameters, such as the shape of the stimulation waveform, amplitude of current in mA (or amplitude of voltage in V), pulse width in microseconds, frequency in Hz, and anodic or cathodic stimulation.

Despite advances in medical technology, the existing sacral nerve stimulation (SNS) methods, systems, and devices still have various shortcomings. Additionally, such devices have not been successfully utilized in treatment of certain diseases, such as inflammatory diseases. As such, there exists a need for more advanced devices as well as methodologies for utilizing such devices to treat a wider range of diseases.

### SUMMARY OF THE INVENTION

The present invention is based on an innovative method of closed-loop SNS for treatment of a disease, such as an inflammatory disease. A device for SNS is also provided in which the device is configured to electrically stimulate the sacral nerve and automatically adjust the electrical stimulation based on detected physiological parameters of the patient.

Accordingly, in one aspect, the invention provides a device for SNS. The device includes: an implantable lead having a plurality of electrodes; a pulse generator coupled to the lead and configured to generate electrical pulses to be delivered to a subject through the electrodes; a computer memory module containing instructions encoding a control algorithm for controlling generation of the electrical pulses as part of a SNS therapy for the subject; and a computer processor module configured to execute the instructions, wherein the device is configured to alter one or more parameters of the electrical pulses in response to a physiological parameter of the subject. In embodiments the device includes an external or implantable sensor for detection of parameters, such as biomarkers and neural signals. In embodiments the device includes an electronic module for processing the detected parameters.

In another aspect, the invention provides a method of treating a disease or disorder in a subject via SNS. The method includes stimulating the sacral nerve via successive applications of an electrical stimuli, the stimuli including an electrical pulse; detecting a physiological parameter with each successive application of the electrical stimuli; and adjusting the applied electrical stimuli based on the detected physiological parameter. In embodiments, the method further includes stimulating the vagal nerve.

In a related aspect, the invention provides a method of treating an inflammatory disease or disorder in a subject via SNS. The method includes stimulating the sacral nerve, or the sacral nerve and the vagal nerve, by administering an electrical stimuli, wherein the electrical stimuli has a frequency of between about 0.5 to 10 Hz, thereby treating the inflammatory disease or disorder. In embodiments, the method further includes administering electroacupuncture. In various embodiments, the sacral nerve, and optionally the vagal nerve, is stimulated by administering an electrical stimuli having a frequency of about 5 Hz, a pulse width of about 0.5 ms and an amplitude of between about 0.2 to 5 mA. In one embodiment, the electrical stimuli is administered as a series of discrete electrical pulses, each pulse being applied for a duration of about 10 seconds, about every 90 seconds for up to 180 minutes.

In yet another aspect, the invention provides a method of neuromodulation. The method includes stimulating the sacral nerve, or the sacral nerve and the vagal nerve, by administering an electrical stimuli, wherein the electrical stimuli has a frequency of between about 0.5 to 10 Hz, thereby performing neuromodulation. In embodiments, the method further includes administering electroacupuncture. In various embodiments, the sacral nerve, and optionally the vagal nerve, is stimulated by administering an electrical stimuli having a frequency of about 5 Hz, a pulse width of about 0.5 ms and an amplitude of between about 0.2 to 5 mA. In one embodiment, the electrical stimuli is administered as a series of discrete electrical pulses, each pulse being applied for a duration of about 10 seconds, about every 10 seconds for up to 180 minutes.

### BRIEF DESCRIPTION OF THE FIGURES

The invention will be better understood from a reading of the following detailed description taken in conjunction with the drawings in which like reference designators are used to designate like elements.

FIG. 1 is a graphic representation depicting data in relation to disease activity index in rats with different treatments.

FIG. 2 is a graphic representation depicting data related to macroscopic scores in different groups of rats. The macroscopic score was increased with trinitrobenzene sulfonic acid (TNBS) and reduced with VNS, SNS and SNS+VNS but not sham-ES.

FIG. 3 is a graphic representation depicting data generated in relation to the present invention showing an increase in MPO activity by TNBS treatment and reduction by VNS, SNS and SNS+VNS but not sham-ES.

FIG. 4A is a graphic representation depicting data generated in relation to the present invention. Parasympathetic (HF) and sympathetic (LF) activities were calculated from the spectral analysis of HRV.

FIG. 4B is a graphic representation depicting data generated in relation to the present invention (BL: before TNBS; Day 10: 10 days after TNBS; BL Day 20: baseline before SNS; SNS-during: during SNS on Day 20; SNS-after: right after SNS on Day 20; sympathovagal imbalance was noted on Day 10 but normalized during and after SNS).

FIG. 5 is a graphic representation depicting data in relation disease activity index in rats during and after DSS treatment. Increase in DAI demonstrates the success of intestinal inflammation.

FIG. 6 is a schematic diagram of an IPG. In an embodiment of the invention the device of the invention includes the IPG along with an implantable sensor operable to detect a physiological parameter and a computing module with functionality to process the detected parameter.

### DETAILED DESCRIPTION OF THE INVENTION

The present disclosure provides an innovative device and method for SNS, including SNS for treatment of inflammatory disease. While the general methodology is applicable to a variety of diseases, the present disclosure exemplifies treatment of an inflammatory disease, for example, treatment of inflammation in animal models of IBD.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

Cervical VNS has been clinically used for the treatment of epilepsy and in the method, vagal afferent is believed to be stimulated. A similar method with a lower stimulation frequency has been introduced for the treatment of inflammation such as IBD and rheumatoid arthritis via the cholinergic anti-inflammatory pathway. For the distal gut, in addition to the vagal innervation, there is parasympathetic nerve, called sacral nerve. Clinically, SNS has been introduced for the treatment of dysfunction of bladder and fecal incontinence. However, this is the first disclosure of treatment of inflammation with SNS.

The inventors hypothesized that SNS also activates the cholinergic anti-inflammatory pathway and is also applicable for the treatment of inflammation in the distal gut. Technically, SNS is as feasible as VNS but it may avoid any possible side effects on cardiac functions as the sacral nerve does not directly interact with cardiac vagal nerve. To explore feasibility of SNS for inflammation and to compare the performance between SNS and VNS, preliminary studies were performed and described in the Examples herein.

In one aspect, the present disclosure provides a method and device for closed-loop SNS for treatment of inflammatory diseases. While the device and general methodology is expected to be applicable to a variety of diseases, a specific aim is treatment of IBD. As set forth in the Examples, in a rodent model of intestinal inflammation, an open-loop SNS method was developed and shown to significantly and substantially reduce inflammation, and improve inflammatory cytokines. A number of anti-inflammatory cytokines, such as, but not limited to IL-4, IL-2, IL-10 and IFN were significantly increased with SNS; whereas a large number of pro-inflammatory cytokines, IL-1β, IL-6, IL-13, IL-12, IL-17A, IL-18, MCP-1 and TNF-α were significantly reduced with SNS.

While neuromodulation has been increasingly used for treating various conditions, such as epilepsy, depression, pain and urinary and gastrointestinal disorders, it has not been applied for treating inflammation that affects 5% to 7% of general population. Based on data set forth herein, the proposed method is expected to be an effective therapy for inflammation. The method and device of the invention can be applied to treat other diseases of inflammation and therefore the general clinical and social impact is great. In addition, the closed-loop method proposed represents a cutting-edge technology that may be applicable to other methods of neuromodulation that are mostly, if not all, open-loop. The feedback controlled neuromodulation is designed to obtain best performance in each individual subject as the stimulation is adjusted to achieve the pre-defined target that is a surrogate of inflammation. This is expected to greatly increase the percentage of responders to neuromodulation therapies. Scientifically, the project will reveal important information on impairment in autonomic and enteric neural circuitries associated with inflammation and a possible method to repair the impairment.

As such, in one aspect, the present invention provides a method of treating a disease or disorder in a subject via SNS. In a related aspect, the invention provides a method of treating an inflammatory disease or disorder in a subject via SNS.

As used herein, the term “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the term “administration” or “administering” are intended to include an act of applying or delivering electrical stimuli to cells or tissue, such as a nerve. Typically administration is performed via electrodes which may directly or indirectly contact tissue as described herein.

The method of the invention may be performed in a closed-loop manner in which a physiological parameter from the subject is detected which is in-turn used to modify or vary the treatment. The method generally includes stimulating the sacral nerve via successive applications of an electrical stimuli, the stimuli including an electrical pulse; detecting a physiological parameter with each successive application of the electrical stimuli; and adjusting the applied electrical stimuli based on the detected physiological parameter.

In various embodiments, the vagal nerve may be stimulated along with the sacral nerve, either simultaneously or independently. The vagal nerve may be stimulated using the stimulation parameters that are described herein for performing SNS. Further, the method may also include performing electroacupunture utilizing the stimulation parameters that are described herein for performing SNS.

The present invention further provides a device for SNS. The device generally includes an implantable lead having a plurality of electrodes, a pulse generator coupled to the lead and configured to generate electrical pulses to be delivered to a subject through the electrodes; a computer memory module containing instructions encoding a control algorithm for controlling generation of the electrical pulses as part of a SNS therapy for the subject, and a computer processor module configured to execute the instructions, wherein the device is configured to alter one or more parameters of the electrical pulses in response to a physiological parameter of the subject.

In practice, the stimulation lead of the device is inserted into the body of a patient, and implanted and positioned to stimulate sacral nerves, alone or in combination with other areas of the nervous system. The lead may be implanted via a needle and stylet for minimal invasiveness. Further, positioning of the lead may be aided by imaging techniques, such as fluoroscopy. In some embodiments, a plurality of stimulation leads may be provided, for example, to provide stimulation of the sacral nerve as well as the vagal nerve.

The device delivers neurostimulation to the sacral nerves or other regions of the nervous system known to treat a disease or disorder, including, but not limited to an inflammatory disease or disorder, such as ankylosing spondylitis, psoriasis, psoriatic arthritis, rheumatoid arthritis, constipation, urinary and fecal control disorders, interstitial cystitis, inflammation associated with pelvic floor disorders, inflammatory bowel disease (IBD) including Crohn's disease (CD) and ulcerative colitis (UC), or other disease or disorder, such as dyspepsia, gastroparesis, intestinal motility disorder including pseudo-obstruction or postprandial ileus, and visceral pain.

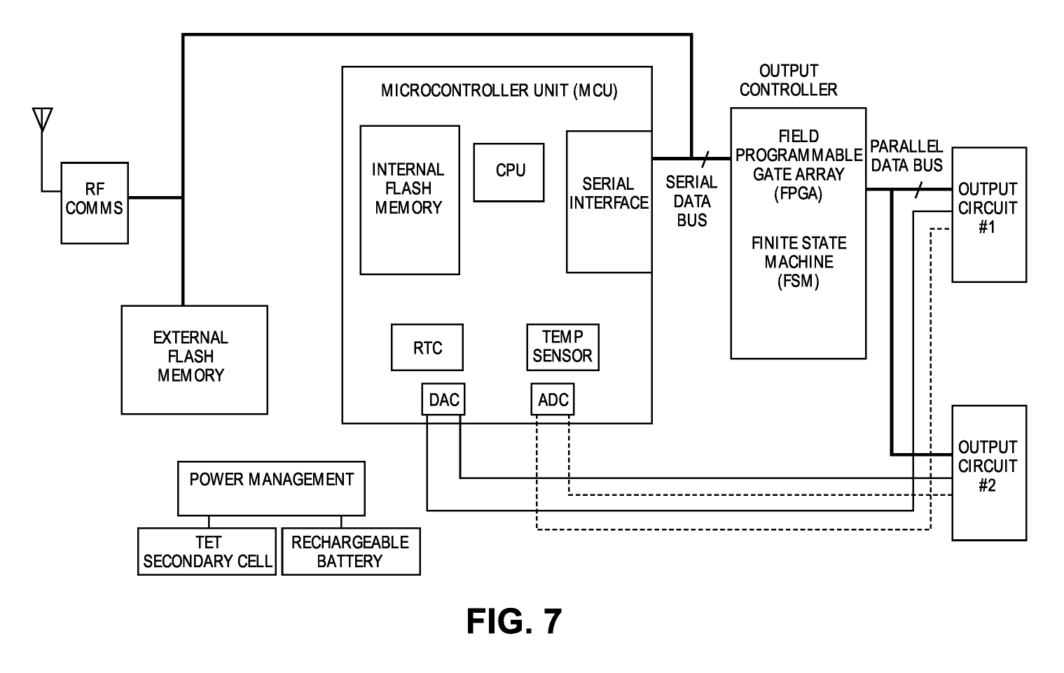

As discussed herein, the device includes at least one lead and an IPG. In some embodiments, the device includes an IPG as in FIG. 6, and delivers neurostimulation therapy to a subject in the form of electrical pulses generated by the IPG. The device may further include a computer memory module containing instructions encoding a control algorithm for controlling generation of the electrical pulses and a computer processor module configured to execute the instructions. The device may further include a sensor for recording a physiological parameter such as a biomarker and an electronics module including functionality for processing the recording from the sensor. In embodiments, the device is a closed-loop device configured to alter one or more parameters of the electrical pulses in response to a physiological parameter of the subject.

The device lead carries one or more stimulation electrodes, for example, 1 to 8 electrodes, to permit delivery of electrical stimulation to the target nerve, such as the sacral nerve. For example, the implantable device may stimulate sacral nerves at the second, third, and fourth sacral nerve positions, commonly referred to as S2, S3, and S4, respectively. In some embodiments, the device may be coupled to two or more leads deployed at different positions relative to the sacral nerves.

In embodiments, the device is configured to deliver electrical stimuli which has been modified in response to a physiological parameter of the subject being treated. This provides a treatment approach that is customized to individual patients. The physiological parameter provides real-time data that is used to optimize efficacy of the treatment protocol.

In some embodiments, a physiological parameter includes a neural signal. Such signals include a neural signal of the sacral nerve, a neural signal of the sacral nerve from a side of the nerve opposing the side which is stimulated and is less than 5 cm from the stimulation electrode, a neural signal of the sacral nerve from a side of the nerve opposing the side which is stimulated and is greater than 5 cm from the stimulation electrode, a neural signal of the vagal nerve, and an electrocardiographic signal generated from a stimulation electrode and a case of an implanted pulse generator. In some embodiments, a physiological parameter includes a biomarker, such as a protein, steroid, hormone or oligonucleotide.

In one embodiment, the biomarker is a protein, such as one or more of G-CSF, Eotaxin, IL-1α, Leptin, Mip-1α, IL-4, IL-1 p, IL-2, IL-6, EGF, IL-13, IL-10, IL-12, IFN, IL-5, IL-17A, IL-18, GRO/KC/CINC-(72), VEGF, FRACTALKING, MIP-2, TNF-α, PANTES, Granulocyte-macrophage colony-stimulating factor (GM-CSF), interferon-inducible protein (IP)-10, monocyte chemoattractant protein (MCP)-1, and monokine induced by IFNγ (MIG).

In another embodiment, the biomarker is an expression product, such as RNA, encoding one or more of G-CSF, Eotaxin, IL-1α, Leptin, Mip-1α, IL-4, IL-1 p, IL-2, IL-6, EGF, IL-13, IL-10, IL-12, IFN, IL-5, IL-17A, IL-18, GRO/KC/CINC-(72), VEGF, FRACTALKING, MIP-2, TNF-α, PANTES, Granulocyte-macrophage colony-stimulating factor (GM-CSF), interferon-inducible protein (IP)-10, monocyte chemoattractant protein (MCP)-1, and monokine induced by IFNγ (MIG).

In another embodiment, the biomarker is a pro-inflammatory cytokine such as one or more of IL-1β, IL-6, IL-13, IL-12, IL-17A, IL-18, MCP-1 and TNF-α.

In another embodiment, the biomarker is an anti-inflammatory cytokine such as one or more of IL-4, IL-2, IL-10 and IFN.

In various embodiments, the sacral nerve, and optionally the vagal nerve, is stimulated by administering an electrical stimuli having a frequency of about 0.5 to 10 Hz, a pulse width of about 0.2 to 1.0 ms and an amplitude of about 0.2 to 5 mA.

In some embodiments, an electrical stimuli is delivered having a frequency of about 5 Hz, a pulse width of about 0.5 ms and an amplitude of between about 0.2 to 5 mA. In one embodiment, electrical stimuli is administered as a series of discrete electrical pulses, each pulse being applied for a duration of about 5-15 seconds, about every 10-100 seconds for up to 5, 10, 15, 20, 30, 40, 50, 60, 70, 80, 90, 100 minutes or more. In one embodiment, electrical stimuli is administered as a series of discrete electrical pulses, each pulse being applied for a duration of about 10 seconds, about every 10 or 90 seconds.

In various embodiments, the IPG of the device of the invention is capable of producing pulse widths from about 100 microseconds to 100 milliseconds in duration in single pulse or pulse train modes. The device may further include a high-gain differential mode bio-amplifier with active analog elliptical band-pass filtering to allow monitoring of low-level electromyographic biomarkers. Filtering is required to minimize the possibility of aliasing in the subsequent analog-to-digital conversion process. Once these signals are converted to the digital domain, they are subjected to further digital filtering to minimize the effects of motion artifacts or ambient sources of electromagnetic interference.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Peripheral Nerve Stimulation: SNS Vs. VNS

Aim

The aim of this preliminary study was to explore the feasibility of SNS for treating inflammation and compare its performance with VNS and the combination of VNS and SNS. It is hypothesized that SNS and VNS are equally effective because the colon is innervated with both vagus nerve and sacral nerves, and that the combination of SNS and VNS results in a synergistic effect, outperforming electrical stimulation of either vagus nerve alone or sacral nerve alone.

Methods

Animal Preparation.

The study was performed in male Sprague-Dawley (SD) rats weighing 250-275 g. For the placement of electrodes for VNS and SNS, an incision was performed to expose the left cervical vagal nerve or right the sacral nerve under anesthesia. One pair of electrodes (stainless steel cardiac pacing wire, Streamline, Medtronic) was gently placed around the left vagal nerve or the right sacral nerve (S3) circumferentially and fixed with surgical knot. The distance between the two electrodes was about 3-5 mm. The electrode connecting wires were first fixed in the muscle layer with sutures and tunneled underneath the skin and externalized at the back of the neck. After the placement of electrodes, the rats were injected with penicillin immediately. Experiments were initiated when the rats were completely recovered (7-10 days) from the surgery.

TNBS-Induced Colonic Inflammation.

The rat was deprived of food for at least 24 h. A 7.5 cm length cannula was inserted into the colon and 2,4,6-trinitrobenzenesulfonic acid (TNBS) was injected at a dose of 22.5 mg per rat in 40% ethanol (total volume, 0.75 ml). To ensure the retention of TNBS within the colon, the rat was maintained in the head-down position following intracolonic administration for 10 min. The control group received the same volume of saline intracolonically.

Experimental Protocols.

In the TNBS model, 64 rats were randomly divided into 8 groups of 8 each: control (no TNBS), and 7 groups with TNBS treatment plus Sham-VNS, VNS, Sham-SNS, SNS, Sham-VNS+SNS and VNS+SNS. respectively. The sham-ES group underwent the same surgical procedure for the placement of electrodes but no electrical stimulation was performed. The VNS, SNS and VNS+SNS groups received electrical stimulation (same parameters for all methods of stimulation: 5 Hz, 0.5 ms pulse width, 10 sec on, 90 sec off, 2.0 mA) 3 hrs daily from 9:00 am-12:00 pm from Days 6 to 15 after the initiation of TNBS treatment.

Measurements and Analyses of Disease Activity Index (DAI).

Behaviorally, inflammation is measured by the DAI that is calculated based on weight loss, stool consistency, and bleeding, which has been commonly used to assess inflammation degree in TNBS or DSS-treated animals. Scores were defined: for weight: 0, no loss; 1, 5%-10%; 2, 10%-15%; 3, 15%-20%; and 4, 20% weight loss; for stool: 0, normal; 2, loose stool; and 4, diarrhea; and for bleeding: 0, no blood; 2, presence; and 4, gross blood. The DAI was scored daily during the entire study.

Measurements of the Plasma Level of Inflammatory Cytokines.

Blood samples (0.8-1.0 ml) were collected in all rats from the tail vein on Days 0 (one day before TNBS injection), 5, 10 and 15 in chilled EDTA and aprotinin tubes in the overnight fasted state. They were then centrifuged at 3000 rpm for 15 min at 4° C. to get plasmas and finally stored at −80° C. For analyzing cytokines, a multiplex sandwich immunoassay from Bio-Plex™ protein array system was used; it contained fluorescence-labeled microspheres conjugated with monoclonal antibodies specific for 27 target cytokines. Plasma samples were thawed and run in duplicates. Antibody-coupled beads were incubated with the plasma sample (antigen) after which they were incubated with biotinylated secondary (detection) antibody before finally being incubated with streptavidin-phycoerythrin. A broad sensitivity range of standards ranging from 1.95-32000 pg/ml were used to help enable the quantitation of a wide dynamic range of cytokine concentrations while still providing high sensitivity. Bound molecules were then read by the Bio-Plex™ array reader which uses Luminex™ fluorescent-bead-based technology with a flow-based dual laser detector with real time digital signal processing to facilitate the analysis of up to 100 different families of color-coded polystyrene beads and allow multiple measurements of the sample ensuing in the effective quantitation of cytokines. Analyses measured levels of G-CSF, Eotaxin, IL-1α, Leptin, Mip-1α, IL-4, IL-1 p, IL-2, IL-6, EGF, IL-13, IL-10, IL-12, IFN, IL-5, IL-17A, IL-18, GRO/KC/CINC-(72), VEGF, FRACTALKING, MIP-2, TNF-α, PANTES, Granulocyte-macrophage colony-stimulating factor (GM-CSF), interferon-inducible protein (IP)-10, monocyte chemoattractant protein (MCP)-1, monokine induced by IFNγ (MIG), and macrophage inflammatory protein (MIP)-1α (source of antibodies: Invitrogen, Carlsbad, Calif.).

Measurement of Myeloperoxidase (MPO) Activity.

The MPO activity of colonic tissue samples was assessed using MPO colorimetric activity assay kit using a previously described procedure. The MPO activity of colonic samples is treated as an index of neutrophil infiltration in the colonic mucosa.

Macroscopic Evaluation of Colonic Damage.

When the rat was sacrificed, the colon was removed from the cecum to anus immediately, opened longitudinally and rinsed with saline to remove fecal residues and then the damage of colon was assessed by the colon mucosal damage index (CMDI) scored on a scale of 0-10 according to the criteria described in previous studies. Briefly, the following scores were applied from Grade 0 (normal appearance) to Grade 8 (major site(s) of damage extending >4 cm along the length of the colon).

Assessment of Autonomic Functions.

The electrocardiogram (ECG) was recorded from a pair of electrodes placed underneath the abdominal skin chronically at baseline (before TNBS) and on Days 5, 10 and 15 after the initiation of TNBS treatment. A heart rate variability (HRV) signal was derived from the ECG recording using previously validated software. The software identifies R waves, calculates R-R interval and interpolates the R-R interval data at 100 Hz, and finally saves the HRV data at a frequency of 8 Hz for analysis. Parameters, including low frequency (LF) component and high frequency (HF) component, were derived from the power spectral analysis of the HRV signal. LF is defined as the area under curve (AUC) in the frequency range of 0.3 to 0.8 Hz, reflecting mainly sympathetic activity. HF is defined as AUC in the frequency range of 0.8 to 4.0 Hz, reflecting purely parasympathetic or vagal activity. LF/HF represents sympathovagal balance.

Results

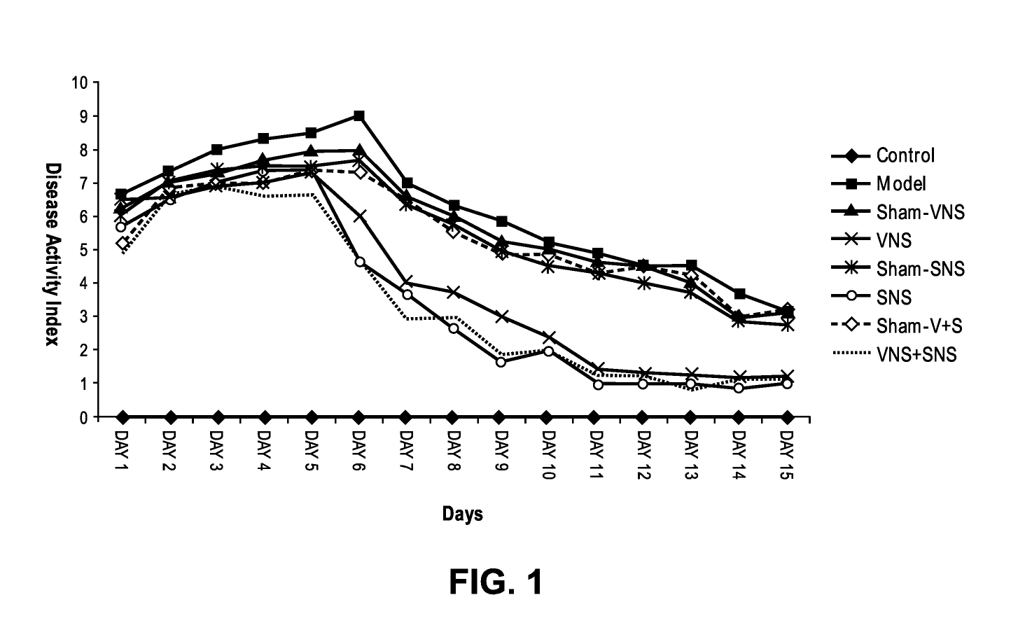

The results of the study are set forth in FIG. 1 to FIG. 4 and summarized as follows.

SNS was as effective as VNS in treating inflammation.

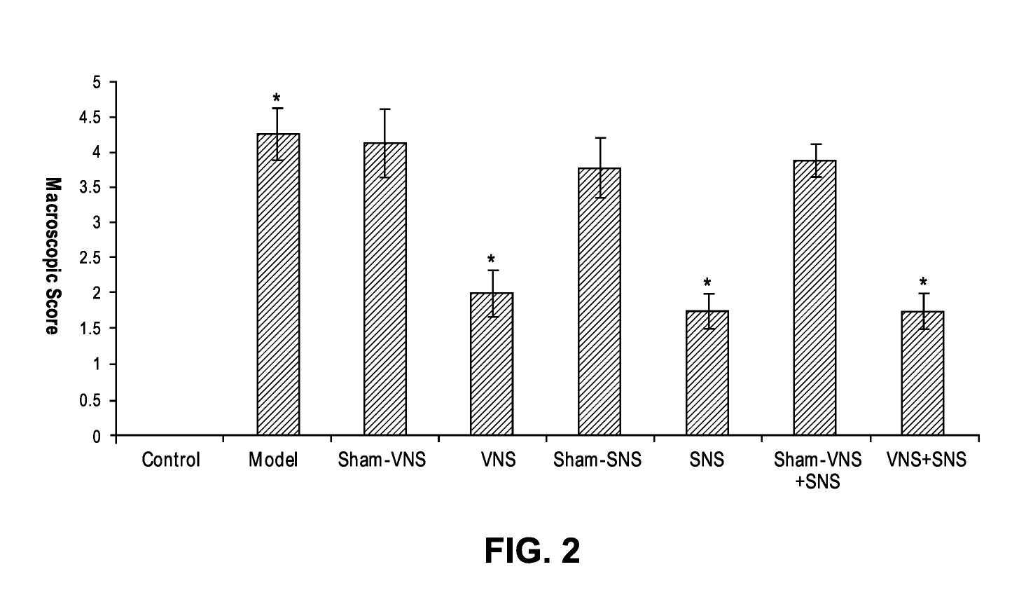

As shown in FIG. 1, the disease activity index was reduced immediately after the initiation of electrical stimulation (Day 5, P<0.01, SNS, VNS or SNS+VNS vs. sham-ES for all points from Day 5 to 15) and more than 50% reduction was noted with all three methods of stimulation; whereas sham-stimulation did not show such an effect. Most importantly, SNS was as effective as VNS and even slightly better than VNS (but not statistically significant). The addition of VNS to SNS did not show any synergistic effect. This is also reflected in the macroscopic scores (FIG. 2)

SNS Improved Cytokine Imbalance.

A number of anti-inflammatory cytokines, IL-4, IL-2, IL-10 and IFN were significantly reduced with the TNBS treatment but increased with chronic SNS; whereas a large number of pro-inflammatory cytokines, IL-1 p, IL-6, IL-13, IL-12, IL-17A, IL-18, MCP-1 and TNF-a were significantly increased with the TNBS treatment but reduced with chronic SNS. Chronic VNS resulted in similar effects on the cytokines: anti-inflammatory, leptin, IL-4, IL-2, EGF, IL-10, IFN and GRO/KC/CINC were significantly decreased with TNBS but significantly increased with VNS; proinflammatory, Eotaxin, GM-CSF, IL-1 a, Mip-1a, IL-1 p, IL-6, IL-13, IL-12, IL-17A, IL-18, MCP-1, FRACTALKING, MIP-2, TNF-a and PANTES were significantly increased with TNBS but reduced with VNS.

SNS Inhibited Myeloperoxidase.

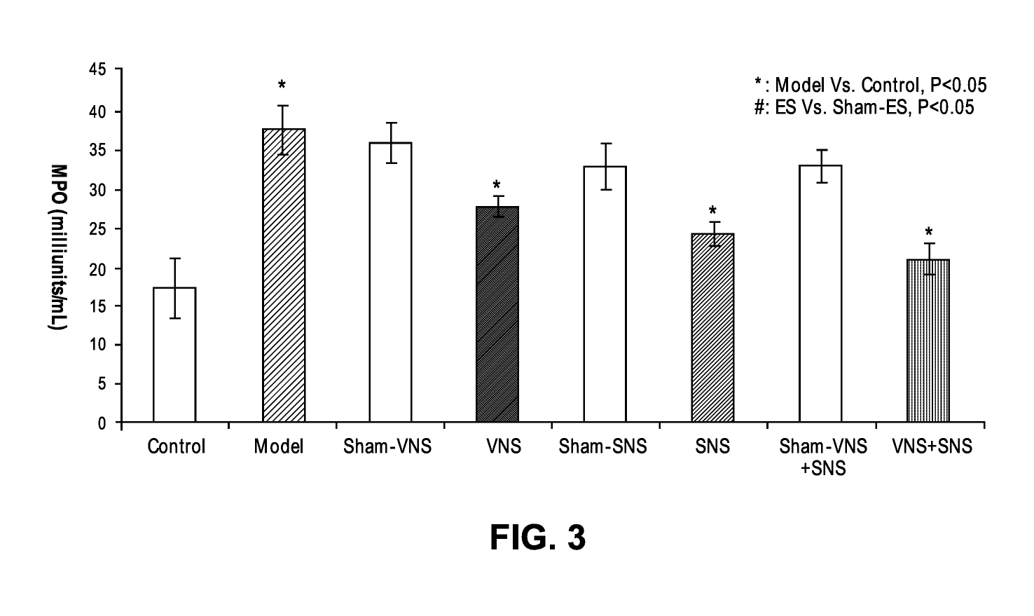

Meanwhile, chronic ES markedly inhibited the MPO activity of the colon tissue, compared with sham-ES group (FIG. 3); the MPO is a sensitive indicator of neutrophil infiltration. Similarly, SNS was as effective as VNS and SNS+VNS in inhibiting MPO activity.

SNS Improved Sympathovagal Balance.

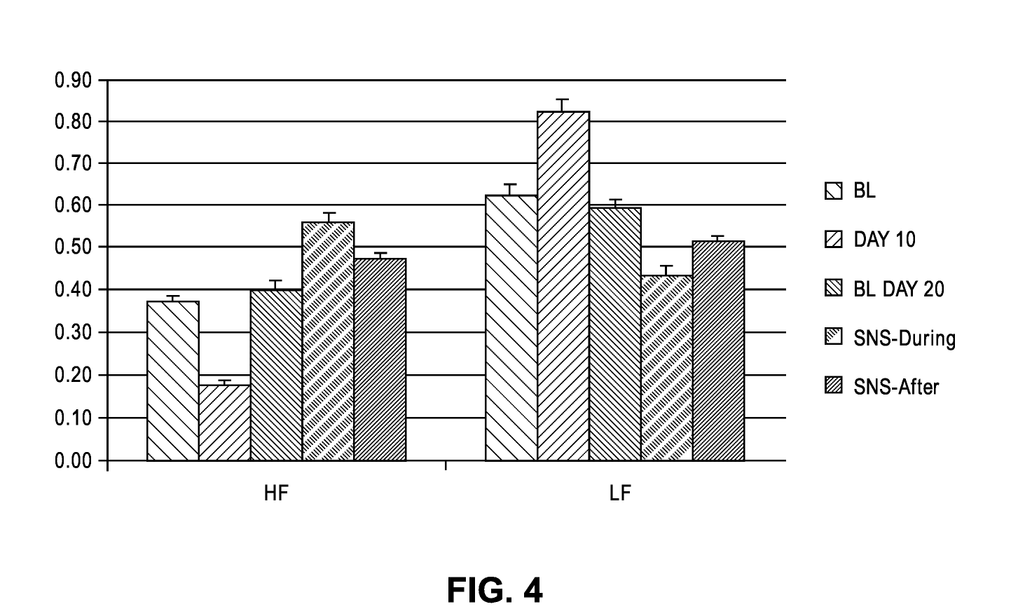

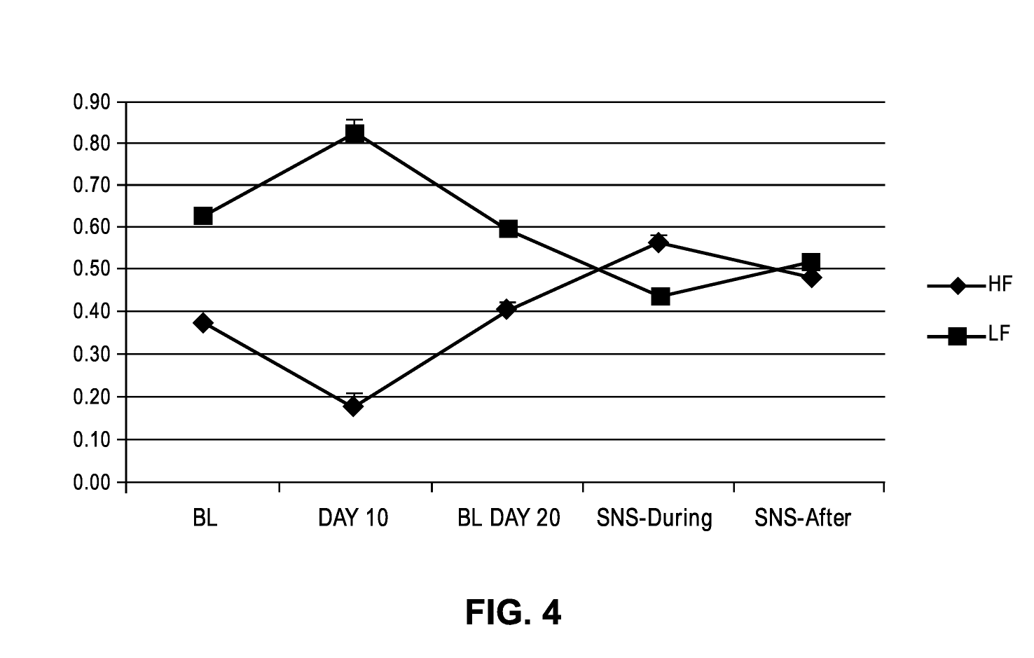

SNS, VNS and SNS+VNS were designed to activate vagal activity and improve sympathovagal balance as inflammation is featured with imbalance of the autonomic function. As expected, all three methods of stimulation showed such an improvement, whereas the sham-stimulation groups did not show such an effect. FIG. 4 presents vagal and sympathetic activities measured from the spectral analysis of the HRV in the SNS group. It is seen that the sympathetic activity was increased but the vagal activity was reduced during the first 10 days after the TNBS treatment. The chronic SNS reduced sympathetic activity and increased the vagal activity (P<0.05, Day 20 vs. Day 10); the similar effects were also noted with acute SNS (P<0.05, Baseline (BL) Day 20 vs. SNS during) and the effects were sustained after termination of stimulation.

From these findings it was determined that SNS activates both afferent and efferent activities in the parasympathetic nerve. This is because the measurements taken in this study reflect cardiac vagal/sympathetic activity but the sacral nerve is not directly linked to the vagal nerve. Accordingly, the effects of SNS on the cardiac vagal/sympathetic must be relayed by the central nervous system via the afferent pathway.

Summary

From this preliminary study it was determined that SNS is as effective as VNS and VNS+SNS in treating intestinal inflammation. A better performance is expected after parameter optimization. The anti-inflammatory effect of SNS is strongly supported by its promising ameliorating effects on inflammatory cytokines. Mechanistically, SNS is shown to improve autonomic imbalance due to inflammation. Since the sacral nerve is not connected with the heart, SNS may be a safer method than VNS with regard to possible side effects on the cardiac function. Accordingly SNS was used to treat inflammation.

### Example 2

### SNS for Treatment of Inflammatory Disease

Aim 1

Alterations in Autonomic-Enteric Neural Circuitries with Inflammation.

Hypothesis

Intestinal inflammation induced by TNBS and dextran sodium sulfate (DSS) results in sympathetic overactivity, impairment in enteric nervous system (ENS) and an increase in macrophages, substance P and vasoactive intestinal peptides (VIP).

The aims of the first series of experiments are as follows. 1) Understand neural (autonomic and enteric nervous systems) pathways involved in the intestinal inflammation and discover alterations and impairment in autonomic and enteric neural circuitries. Neural information are critical for the development of peripheral nerve stimulation and the feedback control of the stimulation. 2) To study alterations in cytokine-producing cell, such as macrophages and to develop an in-vivo biosensor for the assessment of macrophages. This information may be considered as a surrogate of inflammation and thus used to control the proposed SNS. 3) To investigate changes in gastrointestinal peptides/neurotransmitters associated with inflammation, including VIP and substance P.

Methods

1.1 Model of Intestinal Inflammation.

Two commonly used methods for inducing intestinal inflammation will be applied to understand neural and cellular changes under inflammation. The method of TNBS requires only one time intra-colonic administration of TNBS at a dose of 22.5 mg per rat in 40% ethanol (total volume, 0.75 ml). To ensure the retention of TNBS within the colon, the rat will be maintained in the head-down position following intracolonic administration for 10 min. The control group will receive the same volume of saline intracolonically.

In the DDS model, colitis will be induced by feeding the rat with 4% (wt/vol) dextran sodium sulfate (molecular weight 40 kDa) dissolved in drinking water, which will be given ad libitum for ten days. Control rats will receive the same drinking water without DSS.

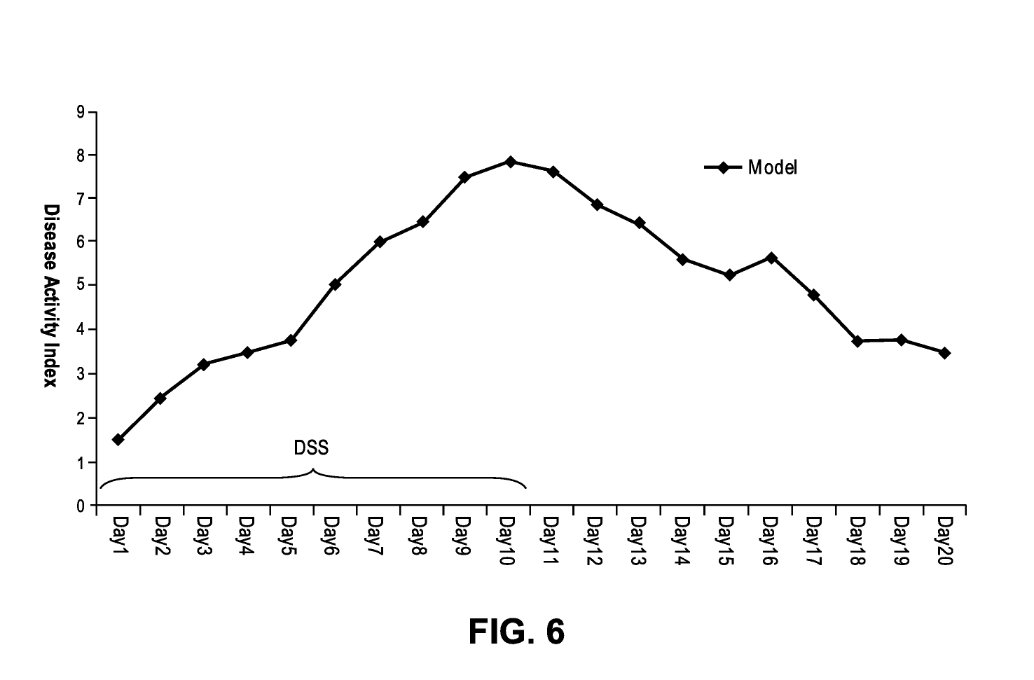

This treatment has been tested in 8 rats and the behavioral score is shown in FIG. 5. As it can be seen that the DAI increased up to 7 and maintained above 3 for at least 20 days. This should give us a sufficiently long window to accomplish our objectives.

In most cases, the study will be performed in normal SD rats. However, with some specific measurements, transgenic mice may be based on the measurement technologies. In these cases, appropriate animals species will be used accordingly.

1.2 Assessment of Inflammation and Cytokines.

Inflammation will be assessed using the methods described in the preliminary study at different time points based on feasibility and necessity. Following assessment methods will be used: 1) DAI measured daily; 2) inflammatory cytokines (27 target cytokines) assessed before and at different time points after the TNBS or DSS treatment; 3) macroscopic assessment of tissue damages in the colon; 4) MPO activity; and 5) microscopic evaluation of colonic damage described as follows: Colonic tissue samples will be fixed in 4% paraformaldehyde for about 24 h and then washed by PBS three times and maintained in PBS overnight. The formalin-fixed colon tissues will be embedded in paraffin wax, and 5 μm specimens will be stained with hematoxylin and eosin (H&E). The following histological parameters will be applied: for inflammatory infiltrate and hyperplasia, grading will be considered as severe (3), moderate (2), mild (1), absent (0); for ulcers, grading will be considered as diffuse glandular disruption or extensive deep ulceration=4, glandular disruption or focal deep ulceration=3, diffuse superficial ulceration=2, focal superficial ulceration=1, absent=0. The three subscores will be summed as the total colitis inflammatory index.

In addition, colon length and spleen weight in the control animals and animals with intestinal inflammation will also be assessed at the end of the study. A preliminary study has demonstrated a reduction of colon length with inflammation and normalization of the length with SNS. Spleen weight was increased with inflammation and normalized with SNS.

1.3 Assessment of Enteric Nervous System.

Inflammation in the distal gut induced by the administration of TNBS is expected to result in impairment in enteric nervous system, such as loss of enteric neurons, excitation of neurons and morphological changes in ENS neurons. The cutting-edge technologies developed by the Co-PI from Cornell University will be used to assess the ENS and immune cells under different conditions: normal, inflammation and after SNS. The technologies and preliminary data are presented as follow.

Currently, there is no technology available to assess the real-time effect of any therapy on the gastrointestinal (GI) tract in live animals. This fundamentally limits our ability to understand and develop any closed-loop therapeutics for inflammation-related or neurologic disorders in the GI. The inventors have developed an innovative platform for simultaneous optical and electrical monitoring of the GI. This platform provides the unique capability of monitoring the status of the ENS and inflammation in the GI with unparalleled spatiotemporal resolution in real-time. This technological capability has the potential to impact the entire gastroenterology community beyond the aims in this proposal.

1.4 Assessment of Autonomic Function.

Studies set forth herein have shown an increase in sympathetic activity and a decrease in vagal activity associated with inflammation, assessed by the spectral analysis of the HRV signal derived from the electrocardiogram (ECG). Spontaneous recovery of inflammation and remission of inflammation with the treatment with VNS/SNS both led to an increase in vagal activity and a decrease in sympathetic activity. Accordingly the autonomic function may serve as a surrogate of inflammation and may be used as a feedback control signal for the closed-loop SNS. The autonomic function will be determined using the following methods: 1) direct in-vivo measurement of parasympathetic activity from the sacral nerves; 2) indirect measurement of autonomic function via the spectral analysis of the heart rate variability signal derived from the ECG; 3) measurements of norepinephrine (released from sympathetic nerves as a neurotransmitter and from adrenal medulla as a hormone) and pancreatic polypeptide (induced by vagal activation) from blood samples.

1.4.1 Direct Assessment of Parasympathetic Activity.

Utilizing this method, one pair of electrodes (same as the SNS wire) will be circumferentially placed around right sacral nerve at a distance of 3-5 mm, using the same method as SNS stimulation electrodes in the preliminary study. The neural signal will be preamplified (200±500, filtered (100±3,000 Hz), further amplified (1,000±2,000), rectified, and time-averaged (time constant 200 milliseconds) (20). The sampling frequency for the raw signal will be at 8 kHz and 10 Hz for the rectified and averaged signal. This recording will be made in live animals via the connecting wires externalized at the back of the neck.

Measurements of norepinephrine (NE) and pancreatic polypeptide (PP): Blood samples will be taken on different days after the administration of TNBS and the plasma level of NE (reflecting sympathetic activation) and PP (indicative of vagal tone) will be assessed using corresponding ELISA kits.

Measurement of cardiac autonomic function: This is a noninvasive measurement and is a surrogate of the autonomic function. The same method described in the Preliminary Study will be used: it is assessed by the spectral analysis of HRV derived from the ECG.

1.5 Measurement of Substance P and VIP.

Substance P and VIP have both been reported to be increased under inflammation. However, it is unclear whether they can be used as surrogates of inflammation. Blood samples on different days (same time points as the other measurements) will be collected in chilled EDTA tubes, centrifuged at 2° C. for 10 minutes, and stored at −70° C. until extraction. Plasma substance P and VIP levels will be determined using commercially available ELISA kits.

Protein and mRNA expression of VIP or SP will be assessed using the following method: all cleaning, full-thickness colonic tissue will be snap-frozen in liquid nitrogen and stored at −80° C. in a freezer. For western blotting, tissues will be lysed and 20 pg protein samples will be separated in 10% sodium dodecyl sulphate polyamide gel electrophoresis (SDS-PAGE) and transferred to a nitrocellulose membrane. The membrane will be g 5% dry milk in PBST for 1 hr, incubated with rabbit antibodies to VIP or SP at 1:1000 dilution overnight at 4° C. and horseradish peroxidase labeled secondary antibody at room temperature for 1 h. Blots will be detected using the enhanced chemiluminescence detection system. β-Actin will be used as internal control.

For RT-PCR, total RNA will be extracted from a fragment of colon tissue of each animal using RNeasy Mini Kit™. Briefly, the tissue will be homogenized in 1000 μl of lysis buffer. 350 μl of dilution buffer will be added to 175 μl of lysate. The sample will be heated at 70° C. for 3 min and centrifuged for 10 min. 250 μl 95% ethanol will be added to cleared lysate and mixed. The lysate will be transferred to column and centrifuged for 1 minute. 600 μl of wash solution will be added and centrifuged for 1 minute. 50 μl of DNase will be applied to column and incubated for 15 minutes at room temperature. After this time, 200 μl of DNase stop solution will be added and centrifuged for 1 min and washed two times. Quantitative real-time PCR of transcription factors including VIP or SP will be performed on a real-time PCR System (Bio-Rad) using SYBR green assay. (3-Actin will be served as endogenous control gene.

Summary of Aim

At the end of this Aim, a better understanding on the changes/impairment in autonomic-enteric neural circuitries, and neural and cellular mechanisms involved in inflammation will be obtained. In addition, advanced in-vivo measurements of biomarkers for inflammation will be made available. These findings will be fundamental for the development of a viable peripheral neuromodulation therapy for inflammation.

Although the invention has been described with reference to the above examples, it will be understood that modifications and variations are encompassed within the spirit and scope of the invention. Accordingly, the invention is limited only by the following claims.

1. A method of treating an inflammatory disease or disorder in a subject comprising stimulating the sacral nerve, or the sacral nerve and the vagal nerve, by administering an electrical stimuli, wherein the electrical stimuli has a frequency of between about 0.5 to 10 Hz, thereby treating the inflammatory disease or disorder.

2. The method of claim 1, wherein the sacral nerve is stimulated.

3. The method of claim 1, wherein the sacral nerve and vagal nerve are stimulated.

4. The method of claim 3, wherein the colon is innervated.

5. The method of claim 3, wherein the vagal nerve is stimulated via stimulation of the cervical nerve.

6. The method of claim 1, wherein the electrical stimuli is applied as a series of discrete electrical pulses.

7. The method of claim 6, wherein the electrical pulse trains are separated by a duration of about 10 to 120 seconds.

8-13. (canceled)

14. The method of claim 1, wherein the electrical stimuli has a pulse width of between about 0.2 to 1.0 ms.

15. (canceled)

16. The method of claim 1, wherein the electrical stimuli has an amplitude of between about 0.1 to 10 mA.

17. The method of claim 1, wherein the electrical stimuli has an amplitude of between about 0.2 to 5 mA.

18. The method of claim 1, wherein the electrical stimuli has a frequency of about 5 Hz.

19-27. (canceled)

28. The method of claim 1, wherein the inflammatory disease or disorder is ankylosing spondylitis, psoriasis, psoriatic arthritis, rheumatoid arthritis, constipation, urinary and fecal control disorders, interstitial cystitis, inflammation associated with pelvic floor disorders, and inflammatory bowel disease (IBD).

29. The method of claim 28, wherein the inflammatory disease or disorder is IBD selected from Crohn's disease (CD) and ulcerative colitis (UC).

30. A method of neuromodulation comprising stimulating the sacral nerve, or the sacral nerve and the vagal nerve, by administering an electrical stimuli, wherein the electrical stimuli has a frequency of between about 0.5 to 10 Hz, thereby performing neuromodulation.

31. The method of claim 30, wherein the sacral nerve is stimulated.

32. The method of claim 30, wherein the sacral nerve and vagal nerve are stimulated.

33. The method of claim 32, wherein the colon is innervated.

34. The method of claim 32, wherein the vagal nerve is stimulated via stimulation of the cervical nerve.

35-80. (canceled)

81. A device comprising:
an implantable lead having a plurality of electrodes;
a pulse generator coupled to the lead and configured to generate electrical pulses to be delivered to a subject through the electrodes;
an implantable sensor operable to detect a physiological parameter and a computing module with functionality to process the detection;
a computer memory module containing instructions encoding a control algorithm for controlling generation of the electrical pulses as part of a sacral nerve stimulation therapy for the subject; and
a computer processor module configured to execute the instructions, wherein the device is configured to alter one or more parameters of the electrical pulses in response to a physiological parameter of the subject.

82-98. (canceled)
